(19) United States
(12) Patent Application Publication
(10) Pub. No.: US 2023/0364208 A1
(43) Pub. Date: Nov. 16, 2023

(54) EFFECTIVE DOSAGE OF RECOMBINANT SERPIN-FC FUSION PROTEIN FOR USE IN A METHOD OF TREATING AAT DEFICIENCY IN A SUBJECT

(71) Applicant: InhibRx, Inc., La Jolla, CA (US)

(72) Inventors: James KALABUS, La Jolla, CA (US); Sharvari BHAGWAT, La Jolla, CA (US)

(21) Appl. No.: 18/318,425

(22) Filed: May 16, 2023

Related U.S. Application Data
(60) Provisional application No. 63/492,692, filed on Mar. 28, 2023.
(60) Provisional application No. 63/342,264, filed on May 16, 2022.

(51) Int. Cl. A61K 38/57; A61K 9/00; A61K 47/26; G01N 33/68

(52) U.S. Cl. CPC A61K 38/57; A61K 9/0019; A61K 47/26; G01N 33/6893; G01N 2333/8125; G01N 2333/96433; G01N 2800/52

(57) ABSTRACT

The present application relates to an effective dosage of an aqueous solution comprising an engineered AAT-Fc fusion dimeric protein for use in a method of treating or alleviating a symptom associated with aberrant serine protease activity in a subject in need thereof.

### RELATED APPLICATIONS

This application claims priority to, and the benefit of, U.S. Provisional Application No. 63/342,264, filed May 16, 2022; and U.S. Provisional Application No. 63/492,692, filed Mar. 28, 2023, the contents of which are hereby incorporated by reference in their entirety.

### INCORPORATION BY REFERENCE OF SEQUENCE LISTING

The Sequence Listing XML associated with this application is provided electronically in XML file format and is hereby incorporated by reference into the specification. The name of the XML file containing the Sequence Listing XML is “INHI-044_001US_SeqList_ST26”. The XML file was created on May 16, 2023, and is 5,021 bytes in size.

### FIELD OF INVENTION

This invention relates to aqueous solutions comprising effective dose ranges of human a AAT-lgG Fc fusion protein and interval of administration of the AAT-lgG Fc fusion protein for treatment of AAT deficiency.

### BACKGROUND

Alpha-1 Antitrypsin Deficiency (AATD) is an under-diagnosed genetic disease affecting an estimated 100,000 patients in the US. It is characterized by insufficient levels of AAT causing emphysema, loss of lung function, and decreased life expectancy. Based on biochemical efficacy, plasma-derived AAT (pdAAT) therapy was approved in the 1980s and is administered weekly to maintain serum AAT concentrations above 11 µM, which is below the normal range. In addition, AAT therapy has been reported to downmodulate inflammation and is being explored to suppress inflammatory diseases and disorders. Since then, little progress has been made with new treatment modalities and cost/supply of pdAAT remains a challenge. A wide range of engineered antibody proteins have been developed, including bispecific and trispecific antibodies. A number of engineered proteins have also been developed wherein the Fc, separated from the Fab parts of an antibody molecule (the parts that confer antigen binding specificity) can serve a purpose different from its physiological purpose, in particular, the purpose of extending the in vivo half-life of the engineered protein. WO2013/003641A2 and WO2016/069574A1 disclose engineered human immunoglobulin G (human IgG) fusion proteins that include a serpin polypeptide or an amino acid sequence that is derived from a serpin. Dosage regimens of these engineered proteins for effective treatment and extended half-life, in the subject’s system need to be determined. The present invention solves this need.

### SUMMARY

The present disclosure provides a method of treating or alleviating a symptom associated with aberrant serine protease activity in a subject in need thereof, the method comprising administering to the subject an AAT-Fc fusion protein by infusion at a dose of about 10 to 120 mg/Kg on the first day of treatment and every three or four weeks thereafter, wherein the AAT-Fc fusion protein comprises the amino acid sequence of SEQ ID NO: 1 or comprising an AAT polypeptide of SEQ ID NO: 2 and an Fc polypeptide of SEQ NO: 3.

In some embodiments, the method comprises administering a dose of about 40 to 120 mg/Kg. In some embodiments, the method comprises administering a dose of about 40 to 80 mg/Kg.

In some embodiments, the method comprises administering a dose of about 60 mg/Kg to 120 mg/Kg.

In some embodiments, the method comprises administering a dose of about 80 mg/Kg.

In some embodiments, the method comprises administering a dose of about 120 mg/Kg.

In some embodiments, a subsequent dose is higher than a previous dose. In some embodiments a subsequent dose is lower than a previous dose. In some embodiments a subsequent dose is same as a previous dose.

In some embodiments, the method comprises administering a dose of about 120 mg/Kg on the first day of treatment and every three weeks thereafter.

In some embodiments, the method comprises administering a dose of about 120 mg/Kg on the first day of treatment and every four weeks thereafter.

In some embodiments, the method further comprises: (a) determining the level of serine protease expression or activity in the subject prior to administration of a first dose to obtain a baseline of expression or activity; (b) determining the level of serine protease expression or activity in the subject at a period of time at least three weeks after administering the first or subsequent dose; and (c) administering a subsequent dose of the AAT-Fc fusion protein (e.g., INBRX-101; SEQ ID NO: 1) that is equal to or higher than the previous dose of the AAT-Fc fusion protein when the serine protease expression or activity in the subject is equal to or higher than the baseline level obtained in step (a); or (d) administering a subsequent dose of the AAT-Fc fusion protein that is lower than the previous dose when the serine protease expression or activity in the subject is lower than the baseline level obtained in step (a).

In some embodiments, the method further comprises: (a) determining the level of AAT expression or activity in the subject prior to administration of a first dose to obtain a baseline of expression or activity; (b) determining the level of AAT expression or activity in the subject at a period of time at least three weeks after administering the dose; and (c) administering a subsequent dose of the AAT-Fc fusion protein (e.g., INBRX-101; SEQ ID NO: 1) that is equal to or higher than the previous dose of the AAT-Fc fusion protein when the AAT expression or activity in the subject is equal to or higher than the baseline level obtained in step (a); or (d) administering a subsequent dose of the AAT-Fc fusion protein that is lower than the previous dose when the AAT expression or activity in the subject is lower than the baseline level obtained in step (a).

In some embodiments, the method further comprises: (a) determining the serum AAT level in the subject at a period of time at least three weeks after administering the first or subsequent dose of the AAT-Fc fusion protein to obtain a serum AAT level; and (b) administering a subsequent dose of the AAT-Fc fusion protein that is equal to or higher than the previous dose of the AAT-Fc fusion protein when the serum AAT level in the subject is below the normal range; or (c) administering a subsequent dose of the AAT-Fc fusion protein that is lower than the previous dose when the serum AAT level in the subject is higher than the normal range. In some embodiments of the above methods, the functional AAT levels are determined. In some embodiments of the above methods, the serum AAT level in the subject is below 15 µM or above 50 µM.

In some embodiments, the AAT-Fc fusion protein (e.g., INBRX-101; SEQ ID NO: 1) is in an aqueous solution comprising: about 5 mg/ml to about 100 mg/ml of the AAT-Fc fusion protein comprising the amino acid sequence of SEQ ID NO: 1; about 5 mM Tris; about 150 mM Trehalose; about 100 mM Sucrose; about 100 mM Proline; about 2 mM Methionine; and about 0.1% (w/v) Poloxamer; wherein the pH of the aqueous solution is adjusted to about 7.3 using either hydrochloric acid or sodium hydroxide; and wherein the total ionic strength of the aqueous solution, excluding the contribution of the AAT-Fc fusion protein, is about 4.3 mM.

In some embodiments, the AAT-Fc fusion protein is in an aqueous solution comprising: about 5 mg/ml to about 100 mg/ml of the AAT-Fc fusion protein comprising the amino acid sequence of SEQ ID NO:1; about 50 mM sodium phosphate; about 125 mM sodium chloride; about 2% (w/v) Trehalose dihydrate; and about 0.01% (w/v) polysorbate 20. In some embodiments, the aqueous solution has a pH of about 7.0.

In some embodiments, the aqueous solution comprises about 50 mg/ml of the AAT-Fc fusion protein.

In some embodiments, the subject in need thereof has aberrant serine protease activity associated with a disease or disorder selected from the following: AAT deficiency, emphysema, chronic obstructive pulmonary disease (COPD), acute respiratory distress syndrome (ARDS), allergic asthma, cystic fibrosis, cancers of the lung, ischemia-reperfusion injury, ischemia/reperfusion injury following cardiac transplantation, myocardial infarction, rheumatoid arthritis, septic arthritis, psoriatic arthritis, ankylosing spondylitis, Crohn’s disease, psoriasis, type I and/or type II diabetes, pneumonia, sepsis, graft versus host disease (GVHD), a wound healing disease or disorder, Systemic lupus erythematosus, and Multiple sclerosis.

In some embodiments, the subject has an infection that is selected from bacterial infections, fungal infections, or viral infections.

In some embodiments, the subject is a human.

In some embodiments, the infusion is delivered over a period of about 30-120 minutes. In some embodiments, the infusion is delivered over a period of about 30-60 minutes.

The present disclosure also provides a unit dose vial comprising: about 5 mg/ml to about 100 mg/ml of the AAT-Fc fusion protein comprising the amino acid sequence of SEQ ID NO:1; about 50 mM sodium phosphate; about 125 mM sodium chloride; about 2% (w/v) Trehalose dihydrate; and about 0.01% (w/v) polysorbate 20, optionally wherein the pH is 7.0.

In some embodiments, a unit dose vial comprises about 50 mg/ml of the AAT-Fc fusion protein.

In some embodiments of the aqueous solution aqueous solution of the present disclosure for use, or use, according to any of the methods of the present disclosure, the subject is a human.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety, in particular, U.S. Provisional Application Nos. 63/342,264, filed May 16, 2022; and 63/492,692, filed Mar. 28, 2023; are incorporated by reference herein in their entirety for any purpose. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a graph depicting the increase of functional AAT levels in serum of all the subject in each dose level group administered different single dose levels of INBRX-101 at baseline, maximum AAT level and AAT level at day 21 post-administration, as indicated. Shaded area of the graph represents normal physiological level of AAT in human serum. Functional AAT levels (µM) are indicated on y-axis and the individual INBRX-101 single dose levels (mg/Kg) are indicated on the x-axis.

FIGS. 2A-2B are two graphs depicting dose dependent increase in functional AAT levels following administration of INBRX-101. FIG. 2A is a graph depicting the average increase of functional AAT levels in serum of subjects in each dose level group administered different single dose levels of INBRX-101 at baseline, maximum AAT level and AAT level at day 21 post-administration. FIG. 2B is a graph depicting the average increase of functional AAT levels in serum of subjects in each dose level group administered different single dose levels of INBRX-101, 6 months post detection of functional AAT levels in FIG. 2A. Shaded area of the graph represents normal physiological level of AAT in human serum. Functional AAT levels (µM) are indicated on y-axis and the individual INBRX-101 single dose levels (mg/Kg) are indicated on the x-axis.

FIGS. 3A-3B are two graphs depicting a comparison of dose dependent increase in functional AAT levels following administration of INBRX-101 or Zemaira. FIG. 3A depicts the observed maximum levels (Cmax) and FIG. 3B depicts trough of functional AAT levels, at dose 1, dose 2 and at steady state (baseline), for each dose level of either INBRX-101 and Zemaira. Shaded area of the graph represents normal physiological level of AAT in human serum. Functional AAT levels (µM) are indicated on y-axis and the individual INBRX-101 single dose levels (mg/Kg) are indicated on the x-axis.

FIGS. 4A-4B are two graphs depicting functional AAT levels over time in AATD patients administered 40, 80 or 120 mg/kg INBRX-101 every three weeks. FIG. 4A depicts the functional AAT levels (µM) in patients administered multiple doses in the amounts of 40, 80 and 120 mg/Kg (as indicated), at an interval of 3 weeks. The shaded region represents the normal range of functional AAT in healthy adults. Functional AAT levels (µM) are indicated on y-axis and the individual INBRX-101 dose time points are indicated with arrows on the x-axis. FIG. 4B depicts the baseline levels of functional AAT in plasma (µM) in 65 healthy volunteers and 30 patients of the phase I AAT study, prior to dosing INBRX-101. Functional AAT levels (µM) are indicated on y-axis and the identity of the healthy volunteers and the AAT variant patients (as determined by Mayo Clinic Laboratories using an LC-MS/MS method (A1ALC) are indicated on the x-axis. Box plots show the minimum, lower quartile, median, upper quartile and maximum. The shaded region represents the 5th-95th percentiles of the normal range of functional AAT in healthy MM genotype adults.

### DETAILED DESCRIPTION

The present disclosure is based in part on the surprising discovery that administering an Alpha-1 Antitrypsin (AAT) Fc fusion protein, also referred to herein as INBRX-101, to individuals with an Alpha-1 Antitrypsin Deficiency (AATD) restored alpha-1 antitrypsin to normal levels.

AATD is a genetic disease characterized by insufficient levels of AAT which cause emphysema, loss of lung function, and decreased life expectancy. Current augmentation therapy with plasma-derived AAT requires weekly IV dosing due to its short half-life and aims to maintain patients above a serum AAT target concentration of 11 µM. In contrast, INBRX-101 an engineered recombinant human AAT-Fc fusion protein that has been demonstrated to achieve serum levels of AAT in the normal range above 20 µM over a dosing interval of at least three weeks. INBRX-101, is the first ATT-Fc fusion protein to achieve and maintain serum AAT levels within the normal range (>20 µM) using an extended dosing interval.

Extended dosing intervals of INBRX-101 reduces the frequency of infusions, eliminates lung decline from AATD, and significantly improve patient quality of life. Accordingly, the disclosure provides methods of treating or alleviating a symptom associated with aberrant serine protease activity in a subject by administering to the subject an AAT-Fc fusion protein (e.g., INBRX-101; SEQ ID NO: 1) by infusion at a dose of about 10 to 120 mg/Kg on the first day of treatment and every three or four weeks thereafter. In some embodiments, serum AAT levels (e.g., functional AAT levels) are generally maintained at >20 µM between dosing.

The amino acid sequence of the INBRX-101 monomer is shown below. The AAT polypeptide portion (SEQ ID NO: 2) of the INBRX-101 is underlined with the Met351Glu mutation in bold and italic, and the Met358Leu mutation is bold and italic: and the IgG4-Fc polypeptide portion (SEQ ID NO: 3) of INBRX-101 is italicized, with mutations S228P, L235E, M252Y and M428L indicated in boxes. A GS linker connecting the AAT polypeptide portion and the IgG4-Fc polypeptide portion indicated in bold.

EDPQGDAAQKTDTSHHDQDHPTFNKITPNLAEFAFSLYRQLAHQSNSTNI FFSPVSIATAFAMLSLGTKADTHDEILEGLNFNLTEIPEAQIHEGFQELL RTLNQPDSQLQLTTGNGLFLSEGLKLVDKFLEDVKKLYHSEAFTVNFGDT EEAKKQINDYVEKGTQGKIVDLVKELDRDTVFALVNYIFFKGKWERPFEV KDTEEEDFHVDQVTTVKVPMMKRLGMFNIQHCKKLSSWVLLMKYLGNATA IFFLPDEGKLQHLENELTHDIITKFLENEDRRSASLHLPKLSITGTYDLK SVLGQLGITKVFSNGADLSGVTEEAPLKLSKAVHKAVLTIDEKGTEAAGA EFLEAIPLSIPPEVKFNKPFVFLMIEQNTKSPLFMGKVVNPTQKGSESKY GPPCPPCPAPEFEGGPSVFLFPPKPKDTLYISRTPEVTCVVVDVSQEDPE VQFNWYVDGVEVHNAKTKPREEQFNSTYRVVSVLTVLHQDWLNGKEYKCK VSNKGLPSSIEKTISKAKGQPREPQVYTLPPSQEEMTKNQVSLTCLVKGF YPSDIAVEWESNGQPENNYKTTPPVLDSDGSFFLYSRLTVDKSRWQEGNV FSCSVLHEALHNHYTQKSLSLSLGK (SEQ ID NO:1)

INBRX-101 is produced as a monomer, however in an aqueous solution may form dimers. In some embodiments, the INBRX-101 disclosed herein can be present as a mix of monomeric and dimeric protein. In some embodiments, the monomers of the dimeric protein may be linked to each other by disulfide bridges. In particular, the pair of human immunoglobulin Fc polypeptide or polypeptide that is derived from an immunoglobulin Fc polypeptide are so linked to form a functional Fc domain.

In some embodiments the AAT-Fc fusion protein (e.g., INBRX-101; SEQ ID NO: 1) is administered at a dose (first or subsequent)of about 40 to 120 mg/Kg (e.g., about 40 to 45, 45 to 50, 50 to 55, 55 to 60, 60 to 65, 65 to 70, 70 to 75, 75 to 80, 80 to 85, 85 to 90, 90 to 95, 95 to 100, 100 to 105, 105 to 110, 110 to 115 or 115 to 120 mg/Kg). In some embodiments, the dose is about between 40 to 80 mg/Kg (e.g., about 40 to 45, 45 to 50, 50 to 55, 55 to 60, 60 to 65, 65 to 70, 70 to 75 or 75 to 80 mg/Kg). In some embodiments, the dose is about between 60 to 120 mg/Kg (e.g., about 60 to 65, 65 to 70, 70 to 75, 75 to 80, 80 to 85, 85 to 90, 90 to 95, 95 to 100, 100 to 105, 105 to 110, 1110 to 115 or 115 to 120 mg/Kg). In some embodiments, the dose is about between 60 to 80 mg/Kg (about 60 to 65, 65 to 70, 70 to 75 or 75 to 80 mg/Kg).

In some embodiments, the dose is about 40 mg/Kg. In some embodiments, the dose is about 80 mg/Kg. In some embodiments, the dose is about 120 mg/Kg.

In some embodiments, a dose of about 80 mg/Kg is administered on the first day of treatment and every three weeks thereafter.

In some embodiments, a dose of about 80 mg/Kg is administered on the first day of treatment and every four weeks thereafter.

In some embodiments, a dose of about 120 mg/Kg is administered on the first day of treatment and every three weeks thereafter.

In some embodiments, a dose of about 120 mg/Kg is administered on the first day of treatment and every four weeks thereafter.

In some embodiments, a subsequent dose is higher than a previous dose. In some embodiments a subsequent dose is lower than a previous dose. In other embodiments a subsequent dose is same as a previous dose.

In some embodiments, the period between each dose is the same.

In some embodiments, each subsequent dose is administered three weeks after the previous dose.

In some embodiments, each subsequent dose is administered four weeks after the previous dose.

In some embodiments, one or more subsequent doses are administered 3 weeks after the previous doses, followed by administration of one or more further doses administered four weeks after the previous dose.

The AAT-Fc fusion protein (e.g., INBRX-101; SEQ ID NO: 1) is administered by infusion, i.e., intravenously.

In certain embodiments, the AAT-Fc fusion protein is diluted into an infusion bag comprising a suitable diluent, (e.g., saline, dextrose in water, etc.). Since infusion or allergic reactions may occur, premedication for the prevention of such infusion reactions is recommended and precautions for anaphylaxis should be observed during the antibody administration. In certain embodiments, the infusion is to be administered to the subject over a period of between about 30 minutes and about 4 hours. In certain embodiments, the IV infusion is delivered over a period of about 30-240 minutes, about 30-180 minutes, about 30-120 minutes, or about 30-90 minutes, or over a period of about 30-60 minutes, or over a lesser period, if the subject does not exhibit signs or symptoms of an adverse infusion reaction. In one embodiment, the IV infusion is delivered over a period of about 30-60 minutes. In another embodiment, the infusion is delivered over a period of about 35-55 minutes. In another embodiment, the IV infusion is delivered over a period of about 45 minutes.

Generally, in the above embodiments, administration occurs at the predetermined frequency or periodicity, or within about 1-3 days of such scheduled interval, such that administration occurs 1-3 days before, 1-3 days after, or on the day of a scheduled dose, e.g., once every 3 weeks (± 3 days).

In some embodiments, the method further comprises: (a) determining the level of serine protease expression or activity in the subject prior to administration of a first dose to obtain a baseline of expression or activity; (b) determining the level of serine protease expression or activity in the subject at a period of time at least three weeks after administering the dose in the method of the disclosure; and (c) administering a subsequent dose of the AAT-Fc fusion protein that is equal to or higher than the previous dose of the serine protease fusion protein when the serine protease expression or activity in the subject is equal to or higher than the baseline level obtained in step (a); or (d) administering a subsequent dose of the AAT-Fc fusion protein that is lower than the previous dose when the serine protease expression or activity in the subject is lower than the baseline level obtained in step (a).

In some embodiments, the serine protease activity of the subject in step (a) of the method disclosed herein, is higher than the physiological level of serine protease activity in a normal subject without any symptom or disorder associated with aberrant serine protease expression or activity.

In some embodiments, the serine protease activity of the subject in step (a) of the method disclosed herein, is at least about 1.5 to 2 times higher than the physiological level of serine protease activity in a normal subject without any symptom or disorder associated with aberrant serine protease expression or activity.

A symptom of aberrant serine protease expression or activity, for example a decrease from normal AAT levels, may include for example, shortness of breath, excessive cough with phlegm/sputum production, wheezing, chest pain that increases when breathing in, a decrease in exercise capacity, and a persistent low energy state or tiredness.

In some embodiments, the serine protease activity of the subject can be determined by any conventional method of detecting protein/enzyme activity in tissue of the subject, e.g., kinetic fluorescence assay, spectrophotometric enzyme assays, calorimetric enzyme assays, light scattering enzyme assays and microscale thermophoresis etc.

In some embodiments, the serine protease expression of the subject in step (a) of the method disclosed herein, is higher than the physiological level of serine protease activity in a normal subject without any symptom or disorder associated with aberrant serine protease expression or activity.

In some embodiments, the serine protease expression of the subject in step (a) of the method disclosed herein, is at least about 1.5 to 2 times higher than the physiological level of serine protease activity in a normal subject without any symptom or disorder associated with aberrant serine protease expression or activity. The normal physiological level of the exemplary serine protease, neutrophil elastase (NE) in human plasma is approximately 32 to 56 µg/L. In some embodiments, in a subject with aberrant serine protease expression, the level of the exemplary serine protease, NE can be at least about 48 to 112 µg/L (e.g., about 48 to 52, 52 to 56, 56 to 60, 60 to 66, 66 to 70, 70 to 74, 74 to 78, 78 to 82, 82 to 86, 86 to 90, 90 to 96, 96 to 100, 100 to 104, 104 to 108 or 108 to 112 µg/L).

In some embodiments, the serine protease expression of the subject can be determined by any conventional method of detecting protein/enzyme expression in tissue of the subject, e.g., electrochemiluminescence, chemiluminescence, enzyme linked immune-assay (ELISA), western blot, flow cytometry, mass spectrophotometry etc.

Serine proteases are catalytic enzymes produced by the liver in response to pathological conditions like infections. Alpha-antitrypsin (AAT) is a serine protease inhibitor, which regulates the activity of serine proteases, e.g., neutrophil elastase (NE). Therefore, the level of serine protease activity is inversely proportional to the level of AAT expression and activity. A subject having higher than the physiological level of serine protease activity in a normal subject without any symptom or disorder associated with aberrant serine protease expression or activity, can also have AAT that is lower than the physiological level of serine protease activity in a normal subject without any symptom or disorder associated with aberrant serine protease expression or activity.

In some embodiments, the method further comprises: (a) determining the level of AAT expression or activity in the subject prior to administration of a first dose to obtain a baseline of expression or activity; (b) determining the level of AAT expression or activity in the subject at a period of time at least three weeks after administering the dose in the method of the present disclosure to obtain a baseline level of AAT expression or activity; and (c) administering a subsequent dose of the AAT-Fc fusion protein that is equal to or higher than the previous dose of the AAT-Fc fusion protein when the AAT expression or activity in the subject is equal to or lower than the baseline level obtained in step (a); or (d) administering a subsequent dose of the AAT-Fc fusion protein (e.g., INBRX-101: SEQID NO: 1) that is lower than the previous dose when the AAT expression or activity in the subject is higher than the baseline level obtained in step (a).

In some embodiments, the method further comprises: (a) determining the serum AAT level in the subject at a period of time at least three weeks after administering the first or subsequent dose of the AAT-Fc fusion protein to obtain a serum AAT level; and (b) administering a subsequent dose of the AAT-Fc fusion protein that is equal to or higher than the previous dose of the AAT-Fc fusion protein when the serum AAT level in the subject is below the normal range; or (c) administering a subsequent dose of the AAT-Fc fusion protein that is lower than the previous dose when the serum AAT level in the subject is higher than the normal range. In some embodiments of the above methods, the functional AAT levels are determined. In some embodiments of the above methods, the serum AAT level in the subject is below 15 µM or above 50 µM.

In some embodiments, the AAT activity of the subject in step (a) of the method disclosed herein, is lower than the physiological level of AAT activity in a normal subject without any symptom or disorder associated with aberrant serine protease expression or activity.

In some embodiments, the AAT activity of the subject in step (a) of the method disclosed herein, is at least about 1.5 to 2 times lower than the physiological level of AAT activity in a normal subject without any symptom or disorder associated with aberrant serine protease expression or activity.

In some embodiments, the AAT expression of the subject in step (a) of the method disclosed herein, is lower than the physiological level of AAT expression in a normal subject without any symptom or disorder associated with aberrant serine protease expression or activity.

In some embodiments, the AAT expression of the subject in step (a) of the method disclosed herein, is at least about 1.5 to 2 times lower than the physiological level of AAT expression in a normal subject without any symptom or disorder associated with aberrant serine protease expression or activity. The normal physiological level of AAT in human plasma is approximately 20 to 48 µM (80 to 220 mg/dL). In some embodiments, the level of AAT in the plasma of the subject is about 10 to 32 µM (e.g., about 10 to 12, 12 to 14, 14 to 16, 16 to 18, 18 to 20, 20 to 22, 22 to 24, 24 to 26, 26 to 28, 28 to 30, 30 to 32 µM). AAT deficiency (AATD) is associated with plasma concentrations below 20 µM (e.g., 19 to 20, 18 to 19, 17 to 18, 16 to 17, 15 to 16, 14 to 15, 13 to 14, 12 to 13, 11 to 12 or <11 µM). In some embodiments, a subject has severe AAT deficiency if demonstrated to have a plasma level ≤ 11 µM.

In some embodiments, the AAT activity of the subject can be determined by any conventional method of detecting protein/enzyme activity in tissue of the subject, e.g., kinetic fluorescence assay, spectrophotometric enzyme assays, calorimetric enzyme assays, light scattering enzyme assays, a human anti-neutrophil elastase capacity (ANEC) assay, and microscale thermophoresis etc. (see e.g., Engelmaier A, Weber A. (2022) J Pharm Biomed Anal.; 209:114476).

In some embodiments, the AAT expression of the subject can be determined by any conventional method of detecting protein/enzyme expression in tissue of the subject, e.g., electrochemiluminescence, chemiluminescence, enzyme linked immune-assay (ELISA), western blot, flow cytometry, mass spectrophotometry etc.

In some embodiments, the expression/activity of either AAT or serine protease or both can be determined in subject’s tissue and body fluids, including blood, serum, plasma, sputum, urine, faecal matter, bronchoalveolar lavage, vaginal lavage, semen etc.

In some embodiments wherein the a subsequent dose of the AAT-Fc fusion protein (e.g., INBRX-101: SEQ ID NO: 1) that is equal to or higher than the previous dose of the AAT-Fc fusion protein, a first or initial dose of AAT-Fc fusion protein is about 40 to 80 mg/Kg (e.g., 40 to 45, 45 to 50, 50 to 55, 55 to 60, 60 to 65, 65 to 70, 70 to 75 or 75 to 80 mg/Kg), and a subsequent dose of AAT-Fc fusion protein is about 80 to 120 mg/kg (e.g., 80 to 85, 85 to 90, 90 to 95, 95 to 100, 100 to 105, 105 to 110, 110 to 115 or 115 to 120 mg/Kg). In some embodiments wherein a subsequent dose of the AAT-Fc fusion protein (e.g., INBRX-101: SEQ ID NO: 1) that is equal to or higher than the previous dose of the AAT-Fc fusion protein, a first or initial dose of AAT-Fc fusion protein is about 40 mg/Kg, and a subsequent dose of AAT-Fc fusion protein is about 80 mg/Kg. In some embodiments wherein a subsequent dose of the AAT-Fc fusion protein (e.g., INBRX-101: SEQ ID NO: 1) that is equal to or higher than the previous dose of the AAT-Fc fusion protein, a first or initial dose of AAT-Fc fusion protein is about 80 mg/Kg, and a subsequent dose of AAT-Fc fusion protein is about 120 mg/Kg.

In some embodiments wherein a subsequent dose of the AAT-Fc fusion protein (e.g., INBRX-101: SEQ ID NO: 1) that is equal to or higher than the previous dose of the AAT-Fc fusion protein, a first or initial dose of AAT-Fc fusion protein is about 40 mg/Kg, and a subsequent dose of AAT-Fc fusion protein is about 40 mg/kg. In some embodiments wherein a subsequent dose of the AAT-Fc fusion protein (e.g., INBRX-101: SEQ ID NO: 1) that is equal to or higher than the previous dose of the AAT-Fc fusion protein, a first or initial dose of AAT-Fc fusion protein is about 80 mg/Kg, and a subsequent dose of AAT-Fc fusion protein is about 80 mg/kg. In some embodiments wherein a subsequent dose of the AAT-Fc fusion protein (e.g., INBRX-101: SEQ ID NO: 1) that is equal to or higher than the previous dose of the AAT-Fc fusion protein, a first or initial dose of AAT-Fc fusion protein is about 120 mg/Kg, and a subsequent dose of AAT-Fc fusion protein is about 120 mg/kg.

In some embodiments wherein the a subsequent dose of the AAT-Fc fusion protein (e.g., INBRX-101: SEQ ID NO: 1) that is lower than the previous dose of the AAT-Fc fusion protein, a first or initial dose of AAT-Fc fusion protein is about 80 to about 120 mg/Kg (e.g., 80 to 85, 85 to 90, 90 to 95, 95 to 100, 100 to 105, 105 to 110, 110 to 115 or 115 to 120 mg/Kg), and a subsequent dose of AAT-Fcfusion protein is about 40 to about 80 mg/kg (e.g., 40 to 45, 45 to 50, 50 to 55, 55 to 60, 60 to 65, 65 to 70, 70 to 75 or 75 to 80 mg/Kg).

In some embodiments wherein a subsequent dose of the AAT-Fc fusion protein (e.g., INBRX-101: SEQ ID NO: 1) that is lower than the previous dose of the AAT-Fc fusion protein, a first or initial dose of AAT-Fc fusion protein is about 80 mg/Kg, and a subsequent dose of AAT-Fc fusion protein is about 40 mg/kg. In some embodiments wherein a subsequent dose of the AAT-Fc fusion protein (e.g., INBRX-101: SEQ ID NO: 1) that is lower than the previous dose of the AAT-Fc fusion protein, a first or initial dose of AAT-Fc fusion protein is about 120 mg/Kg, and a subsequent dose of AAT-Fc fusion protein is about 80 mg/Kg. In some embodiments wherein a subsequent dose of the AAT-Fc fusion protein (e.g., INBRX-101: SEQ ID NO: 1) that is lower than the previous dose of the AAT-Fc fusion protein, a first or initial dose of AAT-Fc fusion protein is about 120 mg/Kg, and a subsequent dose of AAT-Fc fusion protein is about 40 mg/Kg.

In some embodiments, the subsequent dose is administered every three weeks, every 4 weeks, every 5 weeks, every 7 weeks, every 8 weeks (or 2 months), every 10 weeks, every 12 weeks, every 13 weeks, every 14 weeks, every 15 weeks, every 16 weeks (or 4 months), every 17 weeks, every 18 weeks, every 19 weeks, every 20 weeks (or 6 months), every 21 weeks, every 22 weeks, every 23 weeks, every 24 weeks, every 25 weeks, every 26 weeks, every 27 weeks, every 28 weeks, every 29 weeks, every 30 weeks, every 31 weeks, every 32 weeks (or 8 months), every 33 weeks, every 34 weeks, every 35 weeks, every 36 weeks, every 37 weeks, every 38 weeks, every 39 weeks, every 40 weeks, every 41 weeks, every 42 weeks, every 43 weeks, every 44 weeks, every 45 weeks, every 46 weeks, every 47 weeks or every 48 weeks (or 12 months), after the first or previous dose.

In some embodiments, the subsequent dose is administered every three weeks after the first or previous dose. In some embodiments, the subsequent dose is administered every four weeks after the first or previous dose.

In some embodiments, the subject in need thereof has aberrant serine protease activity associated with a disease or disorder selected from the following: AAT deficiency, emphysema, chronic obstructive pulmonary disease (COPD), acute respiratory distress syndrome (ARDS), allergic asthma, cystic fibrosis, cancers of the lung, ischemia-reperfusion injury, ischemia/reperfusion injury following cardiac transplantation, myocardial infarction, rheumatoid arthritis, septic arthritis, psoriatic arthritis, ankylosing spondylitis, Crohn’s disease, psoriasis, type I and/or type II diabetes, pneumonia, sepsis, graft versus host disease (GVHD), a wound healing disease or disorder, Systemic lupus erythematosus, and Multiple sclerosis.

In some embodiments, the subject has an infection that is selected from bacterial infections, fungal infections, or viral infections. In some embodiments, the subject is a mammal. In some embodiments for use, or use, according to any of the methods of the present disclosure, the subject is a human, a rodent, a feline, a canine, a bovine, an equine, a camelus or a mammalian subject. In some embodiments for use, or use, according to any of the methods of the present disclosure, the subject is a human.

### Pharmaceutical Compositions

The engineered AAT-Fc fusion protein of the invention (e.g., INBRX-101; SEQ ID NO: 1) can be further incorporated into pharmaceutical compositions suitable for administration. Such compositions typically comprise an AAT-Fc fusion protein (e.g., INBRX-101; SEQ ID NO: 1) and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington’s Pharmaceutical Sciences, a standard reference text in the field, which is incorporated herein by reference. Preferred examples of such carriers or diluents include, but are not limited to, water, saline, ringer’s solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, epidural, subcutaneous, intramuscular, intradermal, subcutaneous, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR® EL (CrEL) (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an AAT-fusion protein (e.g., INBRX-101; SEQ ID NO: 1) in the required amount in an appropriate solvent with one or a combination of ingredients enumerated herein, as required, followed by filtered sterilization. Generally, dispersions are prepared by incorporating an AAT-Fc fusion protein (e.g., INBRX-101; SEQ ID NO: 1) into a sterile vehicle that contains a basic dispersion medium and the required other ingredients from those enumerated herein. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, pharmaceutical compositions include a unit dose vial. In some embodiments, a unit dose vial comprises: about 5 mg/ml to about 100 mg/ml of the AAT-Fc fusion protein comprising the amino acid sequence of SEQ ID NO:1; about 50 mM sodium phosphate; about 125 mM sodium chloride; about 2% (w/v) Trehalose dihydrate; and about 0.01% (w/v) polysorbate 20.

In some embodiments a unit dose vial comprises about 5 mg/ml to about 100 mg/ml of an AAT-Fc fusion protein (i.e., INBRX-101; SEQ ID NO: 1), about 9.5 mg/ml disodium phosphate heptahydrate; about 0.2 mg/ml monosodium phosphate monohydrate; about 7.26 mg/ml sodium chloride; about 20.0 mg/ml Trehalose dihydrate; and about 0.1 mg/ml polysorbate 20 solution. In some embodiments, a unit dose vial comprising: about 5 mg/ml to about 100 mg/ml of the AAT-Fc fusion protein comprises the amino acid sequence of SEQ ID NO:1; about 9.5 mg/ml disodium phosphate heptahydrate; about 2.0 mg/ml monosodium phosphate monohydrate; about 7.26 mg/ml sodium chloride; about 20.0 mg/ml Trehalose dihydrate; and about 0.1 mg/ml polysorbate 20 solution. In some embodiments, a unit does vial comprises about 50 mg/ml of the AAT-Fc fusion protein. In some embodiment, the contents of a unit dose vial have a pH of about 7.0.

Pharmaceutical compositions include an AAT-Fc fusion protein (e.g., INBRX-101; SEQ ID NO: 1) in an aqueous solution having a pH in the range 7.0 to 8.0 and one or more buffers having at least one ionizable group at a concentration of about 1 to 50 mM; an uncharged tonicity modifier at 50 to 200 mM each; a surfactant at about 0.01 to 2 mg/ml; optionally one or more neutral amino acids at 0 to 300 mM each.

The aqueous solution has a pKa in the range 4.0 to 10.0 and which pKa is within 2 pH units of the pH of the aqueous solution.

The total ionic strength of the aqueous solution excluding the contribution of the AAT-Fc fusion protein (e.g., INBRX-101; SEQ ID NO: 1) is less than 30 mM.

Exemplary buffer or buffers include citrate, histidine, maleate, sulphite, aspartame, aspartate, glutamate, tartrate, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine, N,N′-bis(2-ethanesulphonic acid), phosphate, N,N-bis(2-hydroxyethyl)-2-aminoethanesulphonic acid, 3-[N,N-bis(2-hydroxyethyl)amino]-2-hydroxypropanesulphonic acid, triethanolamine, piperazine-N,N′-bis(2-hydroxypropanesulphonic acid), tris(hydroxymethyl)aminomethane (TRIS), N tris(hydroxymethyl)glycine and N-tris(hydroxymethyl)methyl-3-aminopropanesulphonic acid, and salts thereof, disodium phosphate heptahydrate and monosodium phosphate monohydrate or a combinations thereof.

Exemplary uncharged tonicity modifiers include polyols, sugars (e.g., monosaccharides and disaccharides) and sugar alcohols. In some embodiments, the uncharged tonicity modifier is selected from the group consisting of glycerol, 1,2-propanediol, mannitol, sorbitol, glucose, sucrose, trehalose, PEG300 and PEG400.

The total concentration of the uncharged tonicity modifier, or combination of more than one tonicity modifier, is 50-1000 mM, such as 200-600 mM, 200-500 mM or wherein the total concentration of the uncharged tonicity modifier, or combination of more than one tonicity modifier, is 50-500 mM, such as 100-400 mM, 150-350 mM, 200-300 mM or about 250 mM. In some embodiments, the total concentration of the uncharged tonicity modifier, or combination of more than one tonicity modifier, is 50-150 mM.

In some embodiments the aqueous solution comprises one or more neutral amino acid selected from glycine, methionine, proline, alanine, valine, leucine, isoleucine, phenylalanine, tyrosine, tryptophan, serine, threonine, asparagine, and glutamine.

In some embodiments the total concentration of the one or more neutral amino acids in the aqueous solution is 2 to 100 mM. In some embodiments the total concentration of the one or more neutral amino acids in the aqueous solution is 20 to 600 mM, such as 20 to 500 mM, such as 20 to 400 mM, such as 20 to 300 mM e.g. 50 to 300 mM. In some embodiments, the total ionic strength of the aqueous solution excluding the contribution of the AAT-Fc fusion protein is less than 20 mM.

As used herein, a neutral amino acid is an amino acid the side chain of which does not contain an ionisable group which is significantly ionized (e.g. more than 20% especially more than 50% of the side chain have a minus or plus charge) at the pH of the aqueous solution. Exemplary neutral amino acids are glycine, methionine, proline, alanine, valine, leucine, isoleucine, phenylalanine, tyrosine, tryptophan, serine, threonine, asparagine, and glutamine and in particular the L isomers thereof.

In some embodiments, the aqueous solution comprises a non-ionic surfactant. In some embodiments, the non-ionic surfactant is selected from the group consisting of an alkyl glycoside, a polysorbate, an alkyl ether of polyethylene glycol, a block copolymer of polyethylene glycol and polypropylene glycol (poloxamer), and an alkylphenyl ether of polyethylene glycol. In some embodiments, the non-ionic surfactant is a polysorbate such as polysorbate 20 or polysorbate 80. In some embodiments, the non-ionic surfactant is a block copolymer of polyethylene glycol and polypropylene glycol (poloxamer), such as poloxamer 188.

In some embodiments, the total ionic strength of the aqueous solution excluding the contribution of the AAT-Fc fusion protein is less than 20 mM.

In some embodiments, the pH of the aqueous solution is between about 7.2 and about 7.5 (e.g., 7.2, 7.3, 7.4 or 7.5).

In some embodiments, the aqueous solution comprises a non-ionic surfactant. In some embodiments, exemplary non-ionic surfactants include alkyl glycoside, a polysorbate, an alkyl ether of polyethylene glycol, a block copolymer of polyethylene glycol and polypropylene glycol (poloxamer), and an alkylphenyl ether of polyethylene glycol. In some embodiments, exemplary non-ionic surfactants include a polysorbate such as polysorbate 20 or polysorbate 80. In some embodiments, exemplary non-ionic surfactants include a block copolymer of polyethylene glycol and polypropylene glycol (poloxamer), such as poloxamer 188. In some embodiments, the non-ionic surfactant is present at a concentration of about 0.1 mg/ml to about 10 mg/ml (for example, about 0.1 mg/ml, 0.5 mg/ml, 1 mg/ml, 2 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml).

Polysorbate 20 is a mono ester formed from lauric acid and polyoxyethylene (20) sorbitan in which the number 20 indicates the number of oxyethylene groups in the molecule. Polysorbate 80 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Polysorbate 80 is known under a range of brand names including in particular Tween 80, and also Alkest TW 80. Other suitable polysorbates include polysorbate 40 and polysorbate 60.

In some embodiments, the AAT-Fc fusion protein (e.g., INHBX-101; SEQ ID NO: 1) is in an aqueous solution comprising: about 5 mg/ml to about 100 mg/ml of the AAT-Fc fusion protein protein comprising the amino acid sequence of SEQ ID NO:1; about 5 mM Tris, about 150 mM Trehalose, about 100 mM Sucrose, about 100 mM Proline, about 2 mM Methionine and about 1 mg/ml (0.1% (w/v)) Poloxamer, wherein the pH of the aqueous solution is adjusted to about 7.3 using either hydrochloric acid or sodium hydroxide; and wherein the total ionic strength of the aqueous solution excluding the contribution of the AAT-Fc fusion protein is about 4.3 mM. In some embodiment, the the aqueous solution comprises about 50 mg/ml of the AAT-Fc fusion protein.

In some embodiments, the AAT-Fc fusion protein (e.g., INHBX-101; SEQ ID NO: 1) is in an aqueous solution comprising: about 5 mg/ml to about 100 mg/ml of the AAT-Fc fusion protein comprising the amino acid sequence of SEQ ID NO:1; about 0.6 mg/ml Tris; about 51.3 mg/ml Trehalose; about 34.23 mg/ml Sucrose; about 11.5 mg/ml Proline; about 0.3 mg/ml Methionine; and about 1 mg/ml (0.1% (w/v)) Poloxamer; wherein the pH of the aqueous solution is adjusted to about 7.3 using either hydrochloric acid or sodium hydroxide; and wherein the total ionic strength of the aqueous solution excluding the contribution of the AAT-Fc fusion protein is about 4.3 mM. In some embodiment, the the aqueous solution comprises about 50 mg/ml of the AAT-Fc fusion protein.

In some embodiments, the AAT-Fc fusion protein (e.g., INBRX-101; SEQ ID NO: 1) is in an aqueous solution comprising: about 5 mg/ml to about 100 mg/ml of the AAT-Fc fusion protein comprising the amino acid sequence of SEQ ID NO: 1; about 0.54 mg/ml Tris base; about 0.716 mg/ml Tris hydrochloride 21; about 56.7 mg/ml trehalose dihydrate; about 34.2 mg/ml Sucrose; about 11.5 mg/ml Proline; about 0.3 mg/ml Methionine; and about 1 mg/ml (0.1% (w/v)) Poloxamer; wherein the pH of the aqueous solution is adjusted to about 7.3 using either hydrochloric acid or sodium hydroxide; and wherein the total ionic strength of the aqueous solution, excluding the contribution of the AAT-Fc fusion protein, is about 4.3 mM. In some embodiment, the the aqueous solution comprises about 50 mg/ml of the AAT-Fc fusion protein.

In some embodiments, the AAT-Fc fusion protein (e.g., INHBX-101; SEQ ID NO: 1) is in an aqueous solution comprising about 5 mg/ml to about 100 mg/ml of the AAT-Fc fusion protein comprising the amino acid sequence of SEQ ID NO: 1; one or more buffers being substances at 1 to 40 mM each; one or more tonicity adjuster at 50 to 100 mM each; a surfactant at about 1 mg/ml; and a solvent. In some embodiment, the the aqueous solution comprises about 50 mg/ml of the AAT-Fc fusion protein.

In some embodiments, the AAT-Fc fusion protein is in an aqueous solution comprising: about 5 mg/ml to about 100 mg/ml of the AAT-Fc fusion protein comprising the amino acid sequence of SEQ ID NO:1; about 50 mM sodium phosphate; about 125 mM sodium chloride; about 2% (w/v) Trehalose dihydrate; and about 0.1 mg/ml polysorbate 20. In some embodiments the pH of the aqueous solution is about 7.0. In some embodiment, the the aqueous solution comprises about 50 mg/ml of the AAT-Fc fusion protein.

In some embodiments, the AAT-Fc fusion protein (e.g., INHBX-101; SEQ ID NO: 1) is in an aqueous solution comprising: about 35.4 mM Disodium phosphate heptahydrate, about 1.7 mM Monosodium phosphate monohydrate, about 124.2 mM sodium chloride, about 52.9 mM Trehalose dihydrate, and about 0.1 mg/ml polysorbate 20. In some embodiment, the the aqueous solution comprises about 50 mg/ml of the AAT-Fc fusion protein.

In some embodiments, the AAT-Fc fusion protein (e.g., INHBX-101; SEQ ID NO: 1) is in an aqueous solution comprising: about 35.4 mM Disodium phosphate heptahydrate, about 15.6 mM Monosodium phosphate monohydrate, about 124.3 mM sodium chloride, about 52.9 mM Trehalose dihydrate, and about 0.01% (w/v) polysorbate 20. In some embodiment, the the aqueous solution comprises about 50 mg/ml of the AAT-Fc fusion protein.

In some embodiments, the AAT-Fc fusion protein (e.g., INHBX-101; SEQ ID NO: 1) is in an aqueous solution comprising: about 5 mg/ml to about 100 mg/ml of the AAT-Fc fusion protein comprising the amino acid sequence of SEQ ID NO: 1; about 9.5 mg/ml disodium phosphate heptahydrate; about 0.2 mg/ml monosodium phosphate monohydrate; about 7.26 mg/ml sodium chloride; about 20.0 mg/ml Trehalose dihydrate; and about 0.1 mg/ml polysorbate 20. In some embodiments, the pH of the aqueous solution is 7.0. In some embodiment, the the aqueous solution comprises about 50 mg/ml of the AAT-Fc fusion protein.

In some embodiments, the AAT-Fc fusion protein is in an aqueous solution comprising: about 5 mg/ml to about 100 mg/ml of the AAT-Fc fusion protein comprising the amino acid sequence of SEQ ID NO:1; about 9.5 mg/ml disodium phosphate heptahydrate; about 2.0 mg/ml monosodium phosphate monohydrate; about 7.26 mg/ml sodium chloride; about 20.0 mg/ml Trehalose dihydrate; and about 0.1 mg/ml polysorbate 20 solution. In some embodiments the pH of the aqueous solution is about 7.0. In some embodiment, the the aqueous solution comprises about 50 mg/ml of the AAT-Fc fusion protein.

It should be noted that all references herein to “pH” refer to the pH of an aqueous solution evaluated at 25° C. All references to “pKa” refer to the pKa of an ionisable group evaluated at 25° C. (see CRC Handbook of Chemistry and Physics, 79th Edition, 1998, D. R. Lide). If required, pKa values of amino acid side chains as they exist in a polypeptide can be estimated using a suitable calculator.

In some embodiments, the AAT-Fc fusion protein (e.g., INHBX-101; SEQ ID NO: 1) is in an aqueous solution comprising an osmolarity which is physiologically acceptable and thus suitable for parenteral administration. Thus, the osmolarity of the aqueous solution is suitably 200-500 mOsm/L e.g., about 300 mOsm/L. The aqueous solution is, for example, isotonic with human plasma. In another embodiment, the osmolarity of the aqueous solution is 300-500 mOsm/L e.g., about 400-460 mOsm/L. Aqueous solution may also be hypotonic, or hypertonic, e.g., those intended for dilution prior to administration.

In some embodiments, the aqueous solution may additionally comprise a preservative such as a phenolic or a benzylic preservative. Exemplary preservative is suitably selected from the group consisting of phenol, m-cresol, chlorocresol, benzyl alcohol, propyl paraben and methyl paraben, in particular phenol, m-cresol and benzyl alcohol. The concentration of preservative is typically 10-100 mM, for example 20-80 mM, such as 25-50 mM. The optimal concentration of the preservative in the aqueous solution is selected to ensure the aqueous solution passes the Pharmacopoeia Antimicrobial Effectiveness Test (USP <51>, Vol. 32).

In one embodiment, the active compounds are prepared with carriers that will protect the compound against rapid elimination from the body, such as a controlled release formulation, including implants and microencapsulated delivery systems. Exemplary biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate compositions in a dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

Table 1. Dose levels, number of subject in each dose level group, and number of doses for single administered dose (SAD) and multiple administered dose (MAD) of INBRX-101.

TABLE 1

Dose levels, number of subject in each dose level group, and number of doses for single administered dose (SAD) and multiple administered dose (MAD) of INBRX-101

| Dose Level | Single Administered Dose (SAD) | | Multiple Administered Dose (MAD) | |
|---|---|---|---|---|
| | Number of Subject | Number of Doses | Number of Subjects | Number of Doses |
| 10 mg/kg | N=6 | 1 | NA | None |
| 40 mg/kg | N=6 | 1 | N=6 | 3 |
| 80 mg/kg | N=6 | 1 | N=6 | 3 |
| 120 mg/kg | N=6 | 1 | N=6 | 3 |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### General Methods

### Detection and Quantification of Recombinant Human AAT-Fc Fusion Protein INBRX-101

Detection and quantification of INBRX-101 in human serum was done using Electrochemiluminescence (ECL) assay, where anti-alpha-1 antitrypsin (ANTI-AAT CX2115, InhibRx, #2019011) is coated onto an MSD high-bind plate (ECL capable). The INBRX-101 in standards, QCs, controls and samples was captured onto the coated plate. After a thorough washing of the wells to remove the unbound compounds, Ruthenylated anti-IgG4 (Syneos Health, #94589) is added to the wells. The conjugate binds to the captured INBRX-101. Following the incubation of the detection reagent, the plate is washed, followed by addition of MSD read buffer. The assay plate was then read using an MSD ECL plate reader. The electrochemiluminescence signal generated was relative to the amount of INBRX-101 present in the standards, quality control samples (QCs), controls and samples tested. The concentration of the INBRX-101 is back calculated off of the non-linear regression of the standards. Data were acquired using a Meso Scale Discovery (MSD) Sector S 600. Data capture and analysis were performed in SoftMax Pro Software, version 5.2 or 7.0.1, SoftMax Pro Protocol TM2381.00.

All calculated values, including but not limited to mean concentrations, SD, %CV, and %RE are reported up to three significant figures. Overall statistical calculations (SD, %CV, and %RE) are performed using concentration values that are rounded to three significant figures.

### Assay for the Quantitation of the Function of INBRX-101 Andendogenous AAT in Human Serum by Kinetic Fluorescent Assay.

The function of INBRX-101 and endogenous AAT between 1.00 µM and 8.00 µM in Human Serum was validated using a Kinetic Fluorescent assay. The assay consists of quantitation of the total functional concentration of INBRX-101 and endogenous AAT in human serum. Functional AAT levels in serum samples are determined by their ability to inhibit the activity of NE, measured as a change in the kinetic rate of NE enzymatic activity on its substrate. NE enzymatic activity (assay response) is thus inverse to AAT concentration, where higher AAT concentration results in lower NE enzymatic activity. Due to the nature of the functional assay and the existence of endogenous AAT in serum at measurable concentrations, the method was considered for the PD biomarker analysis of endogenous AAT activity in combination with INBRX-101 drug activity. The assay result was relative-quantitative, reported in µM units of equivalent AAT activity. The calibration curve was generated from plasma derived AAT (pdAAT) as the reference standard.

Furthermore, since endogenous AAT was found at concentrations near or above the assay ULOQ in the target study population, the assay used the surrogate matrix approach where calibrators and quality controls were prepared in AAT depleted serum (stripped matrix). A Pool of AAT depleted human serum (stripped matrix, Lot# BRH1592175 or equivalent) was used as the surrogate matrix to prepare standards, QCs, and to dilute samples. Briefly, standards, QCs and samples containing AAT are diluted to the assay MRD, and then combined with Neutrophil Elastase (NE) in buffer. The mixture was incubated for 30 minutes to allow AAT to irreversibly inhibit NE. Next the samples are added to a read plate with AAPV substrate, and the enzymatic reaction was read for 10 minutes. The resulting kinetic data were reduced and reported in units of Vmax (RFU/sec). AAT concentrations of standards and their resulting Vmax data were used to regress the standard curve against which the concentration of equivalent AAT in samples was determined.

### Example 1 - Evaluation of Safety and Pharmacokinetics of the Recombinant Human AAT-Fc Fusion Protein INBRX-101 in Patients with Alpha-1 Antitrypsin Deficiency

An open-label, international Phase 1 trial designed to assess safety, pharmacokinetics (PK), pharmacodynamics (PD), and immunogenicity of INBRX-101 was conducted. AATD patients were administered single or multiple doses (three consecutive doses every three weeks) of 10, 40, 80 or 120 mg/kg INBRX-101 via IV infusion.

Table 1 shows the dose levels, number of subjects in each dose level group, and number of doses, for single administered dose (SAD) and multiple administered dose (MAD) of INBRX-101.

No drug-related severe or serious adverse events were observed at doses up to and including single doses of 120 mg/kg and multiple doses of 80 mg/kg in 24 patients with AATD. Drug-related adverse events (AE) were predominantly mild with a few moderate events, and all were transient and reversible. The most common drug related adverse events reported were fatigue (n=5), pruritus (n=5), blood pressure increased (n=5), urticaria (n=4), and infusion related reactions (n=2). Infusion related reactions (e.g., pruritus, blood pressure increased, urticaria, infusion related reactions) were transient, mostly mild (Grade 1 per CTCAEv5.0) except for one moderate event (Grade 2) and responded well to symptomatic therapy.

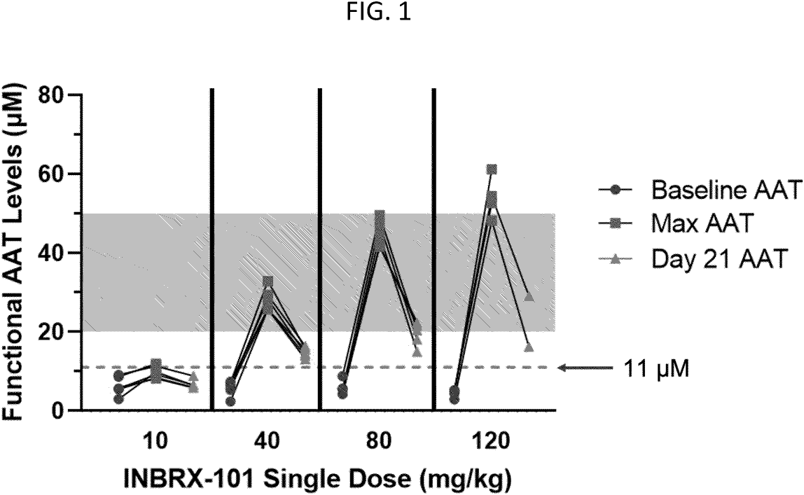

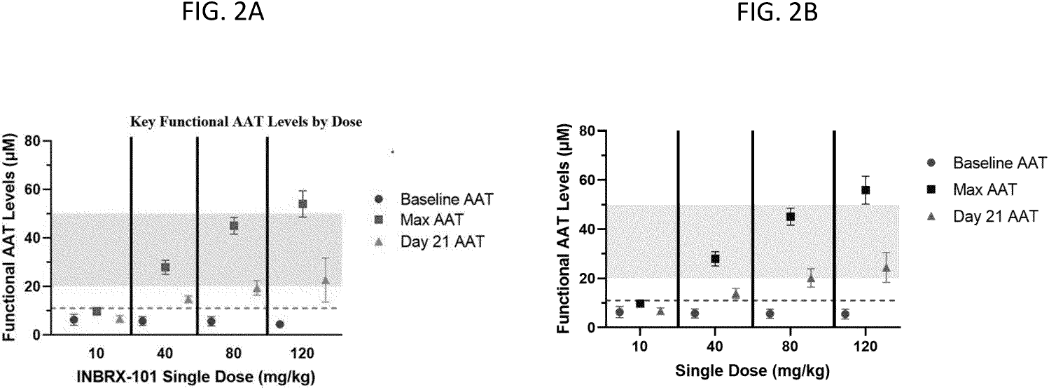

Single dose: Serum antigenic PK and functional AAT levels were assessed in 21 AATD patients. Dose-related increases in maximal and total INBRX-101 exposure were observed across the dose range of 10 to 120 mg/kg, over the baseline (approximately 1 mM). Slight increase in the serum AAT trough was observed over a period of 6 months at the single doses of 80 and 120 mg/Kg (see FIGS. 2A-2B). A higher AAT trough was observed with minimum variability at 120 mg/ml dose, with ⅘ subjects tested showing a trough >20 mM. A terminal half-life of approximately 15 - 19 days was calculated. Functional AAT levels increased rapidly following administration of INBRX-101 and showed a dose response with respect to maximal and 21-day post-dose concentrations (see FIG. 1).

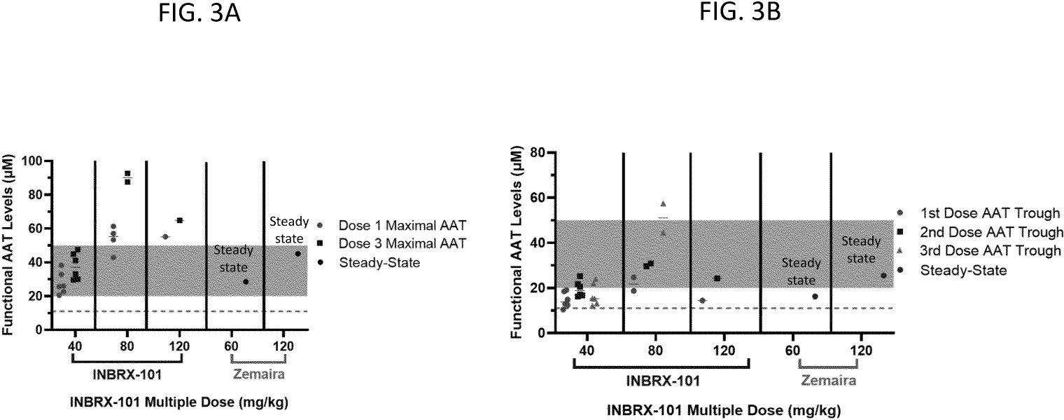

Multiple dose: A multiple dose regimen with each of the multiple dose regimen administered every 3 weeks, being of any one of 40, 80 or 120 mg/Kg. Data over a period of six months is disclosed herein. Preliminary data from multiple doses of 40 mg/kg or 80 mg/kg every three weeks showed accumulation in line with the prolonged half-life. A higher functional AAT level more than baseline, and within the normal physiological range, was observed at 84 days after treatment. Observed maximum levels (Cmax) and trough levels of functional AAT caused by the multiple administered doses exceeded those reported historically for plasma-derived AAT and maintained the AAT levels within the normal physiological level (FIGS. 3A-3B).

Further data from 31 AATD patients of this phase 1 study (26 with the ZZ genotype, 3 with the SZ genotype and 2 with the MZ genotype of the SERPINA1 gene) showed that the treatment was well tolerated with no severe or serious adverse events related to the drug. Drug-related adverse events were predominantly mild and those few that were moderate in severity were all transient and reversible, with minimal or no symptomatic care. No safety-related or PK/PD-related signs of neutralizing anti-drug antibodies were observed.

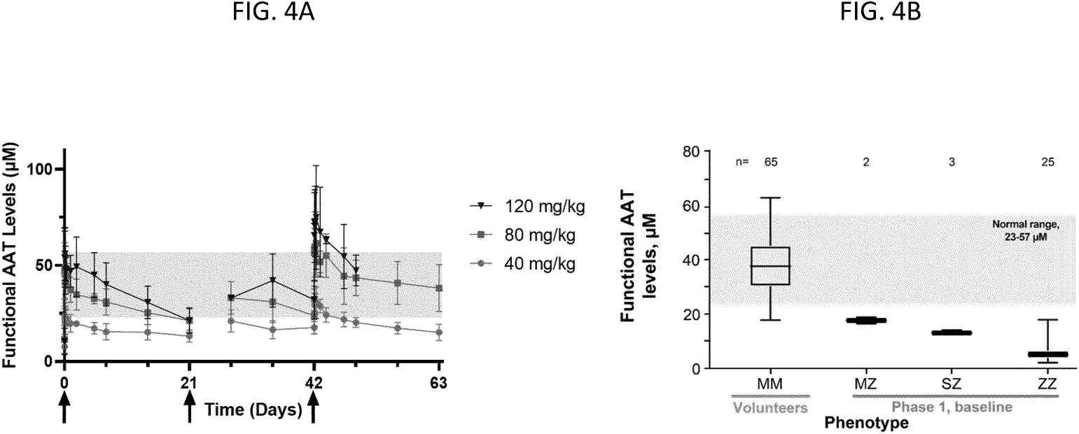

Dose-related increases in maximal and total INBRX-101 exposure occurred across the entirety of the single and multiple ascending dose ranges. Data from the multiple ascending dose cohorts of INBRX-101 at 40, 80 and 120 mg/kg IV every three weeks showed the expected accumulation of functional AAT levels (FIG. 4A). Based on PK modeling, accumulation is expected to continue following subsequent doses and reach a steady-state after approximately 5 to 6 consecutive once every three week doses.

The current standard of care, plasma-derived AAT, dosed once weekly at 60 mg/kg, achieves a Cavg of functional AAT of 17.8 µM over the weekly dosing interval as calculated from steady-state area under the curve (“AUC”) values reported in Stocks et al. BMC Clinical Pharmacology 2010, 10:13. INBRX-101 achieved a mean Cavg of functional AAT of 40.4 µM over the 21-day dosing interval following the third 80 mg/kg dose. Bronchoalveolar lavage fluid (“BALF”) samples processed to date, from two individuals in the 80 mg/kg multiple ascending dose cohort, confirm the presence of INBRX-101 in the lung fluid. Additionally, functional AAT levels were measured in plasma samples from 65 normal MM genotype individuals. This analysis revealed the 5th and 95th percentiles of functional AAT levels in the normal MM genotype individuals were 23 and 57 µM, respectively, with a median of 38 µM (FIG. 4A).

The results described herein showed that INBRX-101 is generally safe and well-tolerated and has shown the potential to maintain normal AAT serum levels, >20 µM, over the entire dosing interval on a dosing schedule of every three weeks with potential for once-monthly dosing. INBRX-101 achieved and maintained normal AAT serum levels above 20 µM and with every three-week or potentially longer dosing intervals. The results disclosed herein also show that the disclosed dosage regimen of INBRX-101 has the potential to maintain patients in a normal range of functional AAT while reducing the total number of infusions annually.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

As used herein, the term “about,” unless indicated otherwise, refers to the recited value, e.g., amount, dose, temperature, time, percentage, etc., ± 10%, ± 9%, ± 8%, ± 7%, ± 6%, ± 5%, ± 4%, ± 3%, ± 2%, or ± 1%.

All patents, patent applications and references mentioned throughout the specification of the present invention are herein incorporated in their entirety by reference.

The invention embraces all combinations of preferred and more preferred groups and suitable and more suitable groups and embodiments of groups recited above.

What is claimed is:

1. A method of treating or alleviating a symptom associated with aberrant serine protease activity in a subject in need thereof, the method comprising: administering to the subject an AAT-Fc fusion protein by infusion at a first dose of about 10 to 120 mg/Kg on the first day of treatment and a subsequent dose of 10 to 120 mg/Kg every three or four weeks thereafter,
wherein the AAT-Fc fusion protein (i) comprises the amino acid sequence of SEQ ID NO: 1 or (ii) comprises an AAT polypeptide of SEQ ID NO: 2 and an Fc polypeptide of SEQ NO: 3.

2. The method of claim 1, comprising administering a first or subsequent dose of about 60 to 120 mg/Kg.

3. The method of claim 1, comprising administering a first or subsequent dose of about 40 to 80 mg/Kg.

4. The method of claim 1, 2, or 3, comprising administering a first or subsequent dose of about 80 mg/Kg.

5. The method of claim 1 or 2, comprising administering a first or subsequent dose of about 120 mg/Kg.

6. The method of any one of claims 1-5, wherein the subsequent dose is higher than the first dose or a previous subsequent dose.

7. The method of any one of claims 1-5, wherein the subsequent dose is lower than the first dose or a previous subsequent dose.

8. The method of any one of claims 1-5, wherein the subsequent dose is same as the first dose or a previous subsequent dose.

9. The method of any one of claims 1, 2, or 5-8, wherein the method comprises administering a first dose of about 120 mg/Kg on the first day of treatment and a subsequent dose every three weeks thereafter.

10. The method of any one of claims 1, 2, or 5-8, wherein the method comprises administering a first dose of about 120 mg/Kg on the first day of treatment and a subsequent dose every four weeks thereafter.

11. The method of any one of claims 1-3 further comprising:
(a) determining the level of serine protease expression or activity in the subject prior to administration of a first dose to obtain a baseline of expression or activity;
(b) determining the level of serine protease expression or activity at a period of time at least three weeks after administering the first dose, or subsequent dose; and
(c) administering a subsequent dose of the AAT-Fc fusion protein that is equal to, or higher than, the previous dose of the AAT-Fc fusion protein when the serine protease expression or activity in the subject is equal to, or higher than, the baseline level obtained in step (a); or
(d) administering a subsequent dose of the AAT-Fc fusion protein that is lower than the previous dose when the serine protease expression or activity in the subject is lower than the baseline level obtained in step (a).

12. The method of any one of claims 1-3, further comprising:
(a) determining the level of AAT expression or activity in the subject prior to administration of a first dose to obtain a baseline of expression or activity;
(b) determining the level of AAT expression or activity at a period of time at least three weeks after administering the first dose, or subsequent dose; and
(c) administering a subsequent dose of the AAT-Fc fusion protein that is equal to or higher than the previous dose of the AAT-Fc fusion protein when the AAT expression or activity in the subject is equal to or lower than the baseline level obtained in step (a); or
(d) administering a subsequent dose of the AAT-Fc fusion protein that is lower than the previous dose when the AAT expression or activity in the subject is higher than the baseline level obtained in step (a).

13. The method of any one of claims 1-3, further comprising:
(a) determining the serum AAT level in the subject at a period of time at least three weeks after administering the first or subsequent dose of the AAT-Fc fusion protein to obtain a serum AAT level; and
(b) administering a subsequent dose of the AAT-Fc fusion protein that is equal to or higher than the previous dose of the AAT-Fc fusion protein when the serum AAT level in the subject is below the normal range; or
(c) administering a subsequent dose of the AAT-Fc fusion protein that is lower than the previous dose when the serum AAT level in the subject is higher than the normal range.

14. The method of claim 13, wherein the functional AAT levels are determined.

15. The method of claim 13 or 14, wherein the serum AAT level in the subject is below about 15 µM or above about 50 µM.

16. The method of any one of claims 1-15, wherein the AAT-Fc fusion protein is in an aqueous solution comprising:
about 5 mg/ml to about 100 mg/ml of the AAT-Fc fusion protein comprising the amino acid sequence of SEQ ID NO: 1;
about 5 mM Tris;
about 150 mM Trehalose;
about 100 mM Sucrose;
about 100 mM Proline;
about 2 mM Methionine; and
about 0.1% (w/v) Poloxamer;
wherein the pH of the aqueous solution is adjusted to about 7.3 using either hydrochloric acid or sodium hydroxide;
wherein the total ionic strength of the aqueous solution, excluding the contribution of the AAT-Fc fusion protein, is about 4.3 mM.

17. The method of any one of claims 1-15, wherein the AAT-Fc fusion protein is in an aqueous solution comprising:
about 5 mg/ml to about 100 mg/ml of the AAT-Fc fusion protein comprising the amino acid sequence of SEQ ID NO:1;
about 50 mM sodium phosphate;
about 125 mM sodium chloride;
about 2% (w/v) Trehalose dihydrate;
and about 0.01% (w/v) polysorbate 20.

18. The method of 17, wherein the aqueous solution has a pH of about 7.0.

19. The method of any one of claims 16-18, wherein the aqueous solution comprises about 50 mg/ml of the AAT-Fc fusion protein.

20. The method of any one of claims 1-19, wherein the subject in need thereof has aberrant serine protease activity associated with a disease or disorder selected from the following: AAT deficiency, emphysema, chronic obstructive pulmonary disease (COPD), acute respiratory distress syndrome (ARDS), allergic asthma, cystic fibrosis, cancers of the lung, ischemia-reperfusion injury, ischemia/reperfusion injury following cardiac transplantation, myocardial infarction, rheumatoid arthritis, septic arthritis, psoriatic arthritis, ankylosing spondylitis, Crohn’s disease, psoriasis, type I and/or type II diabetes, pneumonia, sepsis, graft versus host disease (GVHD), a wound healing disease or disorder, Systemic lupus erythematosus, and Multiple sclerosis.

21. The method of claim 20, wherein the subject has an AAT deficiency.

22. The method of claim 20, wherein the subject has an infection that is selected from a bacterial infection, a fungal infection, or a viral infection.

23. The method of any one of claims 1-22, wherein the subject is a human.

24. The method of any one of claims 1-23, wherein the infusion is delivered over a period of about 30-120 minutes.

25. The method of claim 24, wherein the infusion is delivered over a period of about 30-60 minutes.

26. The method of any one of claims 1-25 wherein the subject in need thereof has a serum AAT level of less than 20 µM prior to the first dose.

27. The method of any one of claims 1-26 wherein the subject in need thereof has a serum AAT level of less than or equal to 11 µM prior to the first dose.

28. A unit dose vial comprising:
about 5 mg/ml to about 100 mg/ml of the AAT-Fc fusion protein comprising the amino acid sequence of SEQ ID NO:1;
about 50 mM sodium phosphate;
about 125 mM sodium chloride;
about 2% (w/v) Trehalose dihydrate; and
about 0.01% (w/v) polysorbate 20.

29. The unit dose vial of claim 28, comprising about 50 mg/ml of the AAT-Fc fusion protein.

30. The unit dose vial of any one of claim 28 or 29, wherein the pH is about 7.0.


SEQUENCE LISTING (3 sequences)

SEQ ID NO: 1 - protein, 625 aa, synthetic construct
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQKGSESKYGPPCPPCPAPEFEGGPSVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 2 - protein, 394 aa, synthetic construct
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAEFLEAIPLSIPPEVKFNKPFVFLMIEQNTKSPLFMGKVVNPTQK

SEQ ID NO: 3 - protein, 229 aa, synthetic construct
ESKYGPPCPPCPAPEFEGGPSVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK